(19) United States
(12) Patent Application Publication
(10) Pub. No.: US 2012/0227737 A1
(43) Pub. Date: Sep. 13, 2012

(54) ANALYTE SENSOR AND METHOD OF USING THE SAME

(75) Inventors: JOHN J. MASTROTOTARO, Los Angeles, CA (US); Rajiv Shah, Rancho Palos Verdes, CA (US); Partha Ray, Simi Valley, CA (US); Kenny J. Long, Simi Valley, CA (US); Andrew C. Hayes, Simi Valley, CA (US); Nandita Patel, Los Angeles, CA (US); Cary D. Talbot, Santa Clarita, CA (US); Bahar Reghabi, Marina Del Rey, CA (US)

(73) Assignee: MEDTRONIC MINIMED, INC., Northridge, CA (US)

(21) Appl. No.: 13/472,117

(22) Filed: May 15, 2012

Related U.S. Application Data
(63) Continuation of application No. 11/634,728, filed on Dec. 6, 2006.

(51) Int. Cl. A61M 5/31; A61M 5/307; A61M 15/00

(52) U.S. Cl. 128/203.14; 604/66

(57) ABSTRACT

An analyte sensor and systems for determining analyte levels in a user's body. The analyte sensor and systems are adapted to be used with single dose medication devices and include a communication system to transmit the communications from the analyte sensor to the user to notify the user of an estimated amount of fluid to deliver to the user's body. More particularly, these apparatuses and methods for use are for providing convenient monitoring of blood glucose levels in determining the appropriate amount of insulin to deliver.

### RELATED APPLICATION DATA

This is a continuation of U.S. patent application Ser. No. 11/634,728, filed Dec. 6, 2006, which is incorporated herein by reference.

### FIELD OF THE INVENTION

Embodiments of this invention relate generally to an analyte sensor and systems and methods for monitoring analyte levels in an individual's body. More particularly, embodiments of this invention relate to apparatuses and methods for providing various features and ways in which to monitor the analyte levels of a multiple daily injection user and to estimate the amount of fluids to be delivered to the user's body.

### DESCRIPTION OF THE RELATED ART

There are analyte sensors used to measure and monitor any type of analyte in the body. For example, diabetic patients use blood glucose (BG) sensors to test their levels of blood glucose daily. Patients with Type 1 diabetes and some patients with Type 2 diabetes use insulin to control their blood glucose level. Diabetics must modify their daily lifestyle to keep their body in balance. To do so, diabetics need to keep strict schedules, including ingesting timely nutritious meals, partaking in exercise, monitoring BG levels daily, and adjusting and dispensing insulin dosages accordingly. Testing of BG levels has been both painful and awkward for the patient. Traditionally, insulin dependent diabetics were required to monitor their BG levels by puncturing a finger tip with a needle. Due to the fact that many patients must conduct such a test multiple times throughout the day to regulate their BG levels, the procedure can be painful and inconvenient.

Typically, patients may employ various calculations based off of the BG levels to determine the amount of insulin to inject. For example, bolus estimation software is available for calculating an insulin bolus. Patients may use these software programs on an electronic computing device, such as a computer, the Internet, a personal digital assistant (PDA), or an insulin delivery device. Insulin delivery devices to be used with these programs generally include infusion pumps and implantable delivery systems. The better bolus estimation software takes into account the patient's present BG level. Presently, a multiple daily injections (MDI) patient must measure his/her blood glucose using a BG measurement device, such as a test strip meter, a continuous glucose measurement system, or a hospital hemacue. BG measurement devices use various methods to measure the BG level of a patient, such as a sample of the patient's blood, a sensor in contact with a bodily fluid, an optical sensor, an enzymatic sensor, or a fluorescent sensor. When the BG measurement device has generated a BG measurement, the measurement is displayed on the BG measurement device. Then the patient may visually read the BG measurement and physically enter the BG measurement into an electronic computing device to calculate a bolus estimate. Finally, once the bolus estimate is calculated, the patient must dispense the insulin bolus or program an insulin delivery device to deliver the bolus into their body. Unfortunately, this process is also cumbersome and is subject to transcribing errors—for example, the patient may inaccurately enter the BG measurement that is displayed on the BG measurement device into the electronic computing device. Thus, if the BG measurement is not entered correctly, the bolus estimate is not accurate, which may lead to the delivery of an inappropriate insulin dose.

Over the years, a variety of analyte sensors have been developed for detecting and/or quantifying specific agents or compositions in a patient's blood, such as BG levels. While the term “analyte” is used herein, it is possible to determine and use other characteristics as well using the same type of system. Gradual developments have allowed these sensors to improve medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994, which are herein incorporated by reference. The recent advancement in medication infusion pump devices appears to have narrowed development of blood glucose sensors toward use with infusion pump devices.

Unfortunately, there are still a significant number of diabetic patients who prefer not to use the infusion pump devices. These patients may be intimidated by the complex technology or wary of the control of the infusion device. Others may not be able to afford the costs associated with these devices. Such patients continue to use multiple daily injections to administer their insulin dosages. Therefore, there is a need for an analyte sensor, such as a blood glucose sensor, that alleviates chances of error in transferring analyte data and can be tailored for use by both MDI users and infusion device users, and includes features that can customize the sensor capabilities for each user. Furthermore, there is a need for an analyte sensor to improve blood glucose control for users of multiple daily injections.

### BRIEF SUMMARY OF THE INVENTION

Embodiments of the invention are generally directed to a sensor device and methods for using the same that involve measuring an analyte level of a user, and factoring in any relevant external factors, to use in estimating a bolus amount of medication and directing the user to dispense that calculated amount. In particular embodiments, the analyte level is blood glucose (BG) level and the medication is insulin.

In accordance with embodiments, there is provided a method of diabetes management that involves receiving a plurality of readings over time from an analyte sensor and processing each of the readings to generate analyte data. The analyte data is used to estimate a bolus amount of medication to be dispensed from a single dose medication device based on the analyte data. In addition, information about external factors may be received to be used in combination with the analyte data to estimate the proper bolus amount to be dispensed. Finally, the method includes displaying an instruction to, for example, a user to deliver the bolus amount.

In further embodiments, there is provided a sensor device for producing data indicative of an analyte level of a user. In one embodiment, the sensor device comprises a sensor adapted to measure an analyte level of a user, sensor electronics coupled to the sensor for receiving the measured analyte level and processing the measured analyte level to generate analyte data, a first transmitter coupled to the sensor electronics and adapted to transmit a communication including the analyte data, a bolus estimator adapted to receive the communication from the first transmitter to estimate a bolus amount of medication to be dispensed from a single dose medication device based upon the analyte data in combination with external factors, and a monitor coupled to the bolus estimator to display a user interface, the monitor having one or more inputs adapted for use by the user to enter and receive information about the external factors, and wherein the user interface displays the estimated bolus amount. The one or more inputs may be any combination of the following, including but not limited to, buttons, keys, tabs, push pads, touch screens and turn dials. The single dose medication device may be, but not limited to, an inhaler, a jet injector, an injection pen, and a syringe.

For user convenience, the sensor device may be integrated in some embodiments into commonly carried accessories, such as a keychain, a watch, a piece of jewelry, an accessory card, a Smartphone or a key fob. The sensor device may include, in yet other embodiments, a memory to store databases of information that are used in estimating the bolus amount, such as for example, user history, food library, drug library or barcode library. These databases may be used to provide “intelligent therapy” for a user in which the sensor device can analyze the user's analyte data in combination with external factors and/or the databases, and suggest recommendations regarding medication dosages and delivery timing or food intake and intake timing.

### BRIEF DESCRIPTION OF THE DRAWINGS

A detailed description of embodiments of the invention will be made with reference to the accompanying drawings, wherein like numerals designate corresponding parts in the figures.

FIG. 1 is a front perspective view of an analyte sensor according to an embodiment of the invention.

FIG. 2 is a block diagram of an analyte sensor according to an embodiment of the invention.

FIG. 3 is a front perspective view of an analyte sensor according to another embodiment of the invention.

FIG. 4 is a diagram of the electronics architecture of an analyte sensor with a custom integrated circuit according to an embodiment of the invention.

FIG. 5A illustrates a flow chart diagram of menu options accessed through the monitor according to an embodiment of the invention.

FIG. 5B illustrates an alternative flow chart diagram of menu options accessed through the monitor according to an embodiment of the invention.

FIG. 6A is a front perspective view of a combined watch and blood glucose sensor according to an embodiment of the invention.

FIG. 6B is a front perspective view of a combined keychain and blood glucose sensor according to an embodiment of the invention.

FIG. 6C is a front perspective view of a combined necklace and blood glucose sensor according to an embodiment of the invention.

FIG. 6D is a front perspective view of a combined accessory card and blood glucose sensor according to an embodiment of the invention.

FIG. 6E is a front perspective view of a combined Smartphone and blood glucose sensor according to an embodiment of the invention.

FIG. 6F is a front perspective view of a combined key fob and blood glucose sensor according to an embodiment of the invention.

### DETAILED DESCRIPTION OF THE INVENTION

In the following description, reference is made to the accompanying drawings which form a part hereof and which illustrate several embodiments of the present inventions. It is understood that other embodiments may be utilized and structural and operational changes may be made without departing from the scope of the present inventions.

Embodiments of the invention are generally directed to sensor device and methods for using the same that involve measuring an analyte level of a user, and factoring in any relevant external factors, to use in estimating a bolus amount of medication and directing the user to dispense that calculated amount. In one embodiment, the sensor device comprises a sensor adapted to measure an analyte level of a user, sensor electronics coupled to the sensor for receiving the measured analyte level and processing the measured analyte level to generate analyte data, a first transmitter coupled to the sensor electronics and adapted to transmit a communication including the analyte data, a bolus estimator adapted to receive the communication from the first transmitter to estimate a bolus amount of medication to be dispensed from a single dose medication device based upon the analyte data in combination with external factors, and a monitor coupled to the bolus estimator to display a user interface, the monitor having one or more inputs adapted for use by the user to enter and receive information about the external factors, and wherein the user interface displays the estimated bolus amount. The one or more inputs may be any combination of the following including, but not limited to, buttons, keys, tabs, push pads, touch screens and turn dials.

One embodiment provides a method of diabetes management that involves, without any particular order, receiving a plurality of readings, from an analyte sensor over a period of time, processing each of the readings to generate analyte data, receiving information about external factors, using the analyte data to calculate a bolus amount of medication as a function of the analyte data in combination with the external factors, and displaying an instruction to, for example, a user, to deliver the bolus amount. In some embodiments, the plurality of readings may be received on a periodic basis. In other embodiments, the plurality of readings may be received on an automatic basis. In yet other embodiments, the plurality of readings may be received in response to user generated request. The bolus amount is delivered through a single dose medication device in particular embodiments. An external factor is one that can affect the calculation of the bolus amount, such as for example, meal consumption, exercise, medication intake, time lapse from the last bolus dispensed, type of medication device used and user sensitivity. The receiving step may involve obtaining two or more actual analyte level readings, of which an average is taken, to yield the final reading. In this manner, each of the plurality of readings generated may be more representative of the actual analyte level. In further embodiments, the displaying step is performed by displaying the monitor on the analyte sensor.

Additional steps may involve automatically tracking the amounts of various diabetes supplies used. For example, the user may input the total amount of lancets, insulin or insulin syringes purchased. The analyte sensor device may then be able to count the number of lancets used or amount of insulin dispensed, and subtract the amount used from the total amount. When supplies run low, the analyte sensor may sound an alarm to alert the user to this fact. There may also be included a step in which an automatic reorder for the supply may be sent.

In embodiments where the analyte sensor is a BG sensor, the included features can be tailored for use by different groups of users, such as multiple daily injection (MDI) users or infusion device users. The sensor may also be a subcutaneous sensor in some embodiments or operate with a lancing device in alternative embodiments. Furthermore, the BG sensor can be used with any variety of therapy/diagnostic devices, such as electronic therapy devices and devices that receive diagnostic information from cardiac and other sensors. Other therapy/diagnostic devices include devices that administer medication. Some examples include but are not limited to single dose medication devices that are suited for daily dispensing of single doses for MDI users. Some common single dose medication devices include syringes, injection pens, or needle-less devices such as jet injectors for sprayable insulin and inhalers for inhalable insulin. These devices may be, in some embodiments, disposable.

The BG sensor is adapted to communicate with such medication devices through wireless or non-wireless methods. The wireless methods include, by no way in limitation, RF, infrared (IR), Bluetooth, ZigBee, and other 802.15 protocols, 802.11 WiFi, spread spectrum communication and frequency hopping communication (CHIPCON chip (available from Chipcon AS in Oslo, Norway)). Embodiments that use multiple frequencies can facilitate better communication because the sensor can continually switch frequencies until it finds the strongest frequency in the area with which to communicate. For example, the CHIPCON chip allows the sensor to do the scanning of the frequencies and then frequency hop to the strongest signal.

In another wireless example, if the user has access to a computer network or phone connection, the user can open communication via the internet to obtain communications from, and send communications to, a nurse, parent, or anyone so desired. A transceiver may be used to facilitate data transfer between a personal computer (PC) and the medication device. Such a communication may also be used by a party, other than the user, to control, suspend, and/or clear alarms. This embodiment could be very useful for a parent to monitor the medication system of a child, or for a physician to monitor the medication system of a patient. As a non-limiting example, further description of a communication station may be found in U.S. Pat. No. 5,376,070, which is herein incorporated by reference. The transceiver may allow patients at home or clinicians in a hospital setting to communicate with the various components of the infusion system via RF telemetry. The transceiver may be used to download device information from the infusion device and sent to the PC when the transceiver is connected in to the serial or parallel port of the PC. In embodiments, the transceiver may derive its power from the PC when the two are connected. In this way, the transceiver conveniently does not require a separate power source. In another embodiment, a cellular phone may be used as a conduit for remote monitoring and programming. In yet other embodiments, the analyte sensor may also act as a transceiver, which would eliminate an extra component.

In the alternative, the communication may be wired, such as in hospital use. In a wired embodiment, there may be a tether physically connecting the infusion device to the sensor. In yet another alternative, the sensor and the medication device could be both wired and wireless—when wired, the two components communicate by wire, and when disconnected, the two components could operate through wireless communication.

Communications between the analyte sensor and its system components, for example, the bolus estimator or single dose medication device, may be performed in a variety of manners. In an embodiment using RF options, there could be employed a “spread spectrum” where a large range of RFs can be used to relay the communication. In another embodiment, changing frequencies can be used so as to pick up whatever frequency is present. This is known as frequency hopping, where the frequency changes periodically to take advantage of all, or substantially all, frequencies available. Another embodiment is one that uses adaptive frequency selection, or Listen Before Talk (LBT), where the devices select the cleanest available channel from those allotted prior to transmitting. In some cases, frequency hopping allows the system to find frequencies that are not being used by other nearby systems and thus avoid interference. In addition, a system may operate in a manner where each component-to-component communication is on a different frequency, or where the delay for each communication is different. Other types of RF, that are not described, may also be used for communication, such as, translation frequency.

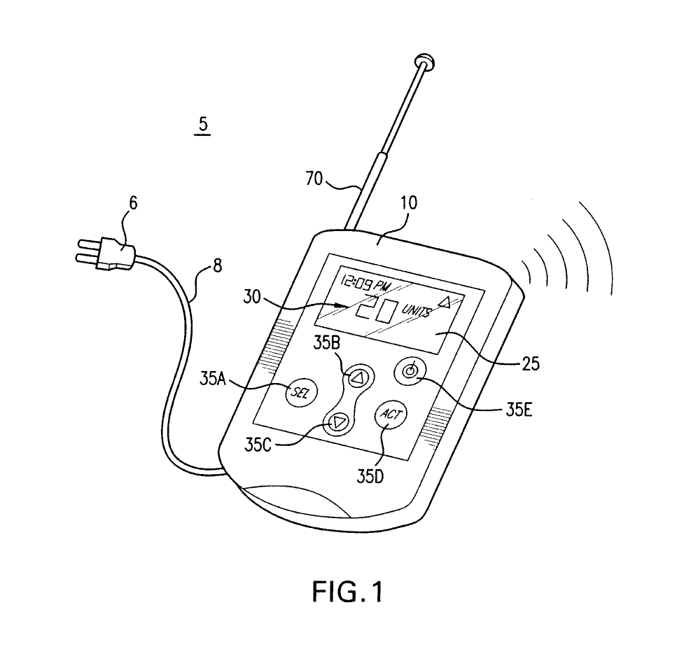

In some embodiments, as shown in FIG. 1, the analyte sensor device 5 includes a housing 10 adapted to be carried by the user and a sensor 6 in communication with the housing 10 that is adapted to measure the analyte level of a user. The sensor 6 may communicate with the rest of the sensor device through a wire 8 or through wireless communication, as discussed above. The analyte sensor device 5 includes a monitor 25 on the housing 10. The monitor 25 displays a user interface which can relay information to the user through a variety of graphical depictions as well as numerical values and text. These graphical depictions may be in the form of a graph, a chart, an extrapolation, a pie chart, a table and the like. The monitor 25 is shown displaying the calculated bolus amount 30 of insulin to be dispensed in units. The monitor 25 also may have one or more inputs 35A, 35B, 35C, 35D and 35E that allow the user to enter information relevant for the bolus estimator to account for in calculating the bolus amount.

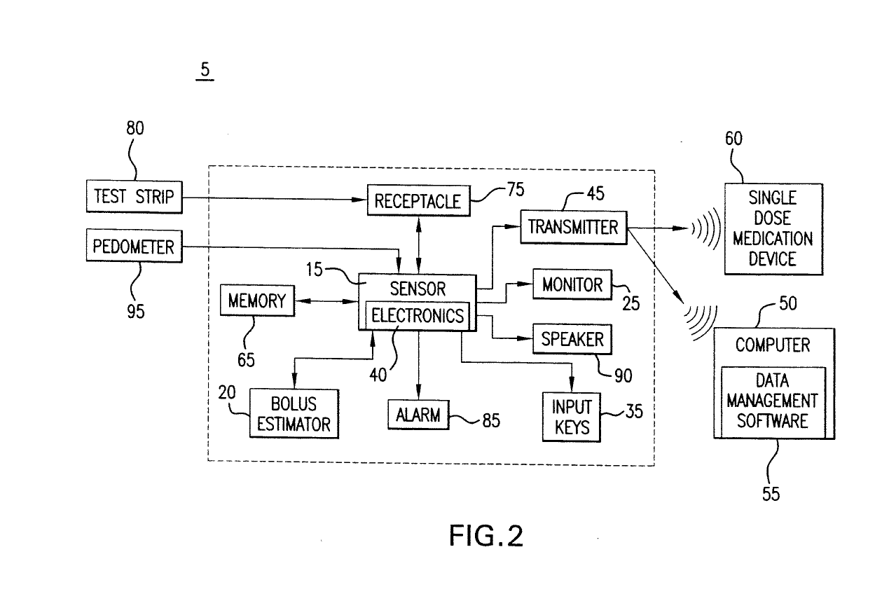

FIG. 2 shows a block diagram of the various components of the sensor device 5. The sensor device 5 comprises a sensor 15 for measuring analyte levels of a user. A bolus estimator 20, coupled to the sensor 15, estimates a bolus amount to be dispensed based on the analyte level and a number of external factors that may be provided by the user. The sensor also includes electronics 40 that can process and convert the measurements into data that can be transmitted or stored. In some embodiments, a transmitter device 45 can wirelessly transmit the data to remotely located devices, such as for example, a computer 50, data management software 55 in a computer 50, or a medication device, like a single dose medication device 60. The analyte sensor device 5 may also include a memory 65 which stores one or more databases for use in calculating the bolus amount. The housing may include, in embodiments, a receptacle 75 coupled to the housing for receiving and testing an analyte from a patient to determine a concentration of the analyte in the patient. The sample containing the analytes may be received by a test strip 80 which is then received by the receptacle 75. Other embodiments may include various types of alarms 85 to alert the user as to, for example, dangerous conditions or an action that needs to be undertaken. A speaker 90 may also be integrated into the sensor device 5 so that audible warnings or notices may be spoken. A speaker 90 may be especially useful for those users that are vision-impaired. Another component that may be included with the sensor device 5 is a pedometer 95 to track how much exercise the user is taking. This exercise amount may be used as an external factor to consider in calculating the bolus amount.

In other embodiments, as seen in FIG. 1, the analyte sensor device 5 may further include a retractable antenna 70 on the housing 10 in embodiments for increasing reception or strength of frequency. The sensor device 5 may also include an indication device that indicates to the user when the bolus amount to be dispensed is calculated. The indication device may be in the form of a visual indication, an audible indication or a tactile indication.

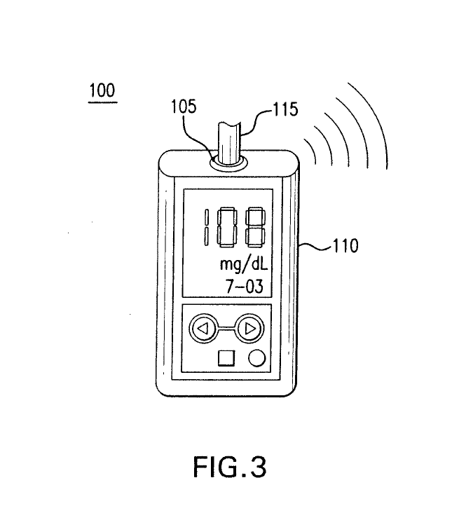

In FIG. 3, a front perspective view is provided of an alternate sensor device 100 that includes a receptacle. In such embodiments, the sensor device receptacle 105 is coupled to the housing 110 for receiving and testing a fluid sample from the user to determine a concentration of the analyte in the user. A test strip 115 that may hold a fluid sample is inserted into the sensor device receptacle 105 for the testing by the analyte sensor device 100. In variations, the sensor device may have a cartridge-like mechanism which loads and presents the strip for testing and then ejects it. In particular embodiments, the sensor device may also include a lancing device coupled to the receptacle for obtaining the sample from the user. For example, the fluid may be blood used to test the blood glucose level of the user.

In alternative embodiments, the analyte sensor may receive communications from or send communications to a therapy/diagnostic device, such as an infusion device, or other components of a medication system. Data compression may be employed to speed up communications. In additional embodiments, the sensor may include accessories such as hand straps to provide convenient handling.

In other embodiments, the analyte sensor includes on the housing a display that may show information requested by the user or an instructed act that was undertaken by the medication device, such as for example, determined concentration of BG levels, trends or graphs. One such system is described and disclosed in U.S. patent application Ser. No. 10/624,177, entitled “System for monitoring Physiological Characteristics,” which is herein incorporated by reference. In one embodiment, the display can show the BG in a variety of ways—as a present BG level or a graphical depiction of BG levels over a continuous period of time. The display may also provide different visual analyses of the analyte levels over different time periods. Furthermore, the display may mimic the display on the medication device. In certain embodiments, whatever is shown on the display of the infusion device or injection device corresponds to that shown and reflected on the display of the analyte sensor. The display may also display information according to communications sent to it from the infusion device or injection device that corresponds to the sensor. For example, when the last bolus was administered, when the last alarm occurred, when the last finger stick was taken, past trends, all alarms that occurred in a time period, calibrations, meals, exercise, bolus schedules, temporary basal delivery, diagnostic information, and the like. Whenever a bolus is being delivered, the medication device can send a message every time a tenth of a unit, or some specified amount, is delivered, to which the user may monitor via the analyte sensor display. In this manner, the user may more conveniently view what is being processed or acted upon in the medication device without removing or adjusting the medication device to check the medication device. In embodiments, the sensor may include one or more input device(s), such as keys, buttons, and the like, on a keypad so that all, or substantially all, viewing and data entry may be performed on the same device without moving the medication device.

There also may be some type of positive mechanism for the analyte sensor if the communication between the analyte sensor and the medication device are interrupted. For example, the mechanism may have the analyte sensor stop displaying its graph in a “time-out” phase for the time the medication device screen is absent or no more data is entered by the user for a period of time. In this case, the medication device operates on the last data that the medication device sent to the analyte sensor to display. In an embodiment, the analyte sensor will display an idle screen during the time-out phase and while the communication between the medication device and the analyte sensor is re-established. The idle screen may remain until the next action is selected by the user. After the time-out phase, the user may press a key to start up the communication again. Once a key is pressed, the analyte sensor will process the key data and the screen will be displayed. The analyte sensor may periodically send signals to the medication device and any other peripheral devices to see if those components are still active on the screen.

In alternative embodiments, there will be a positive confirmation requested prior to displaying graphs. For example, the graphs may be shown in bitmap packets (e.g., bit-by-bit), and if the user will be getting a large number of packets of data, for example 15 packets of data, to show the graph, the user may opt not to confirm. The data is passed from the analyte sensor, which is programmed to display the data, to the medication device. The analyte sensor can operate in graphics description language where data is recognized by the analyte sensor as instructing it on which position to put each line or color and the graphics display would handle determining the resolution that the graph would be displayed in. In some embodiments, the graph may be displayed in three-dimensional format.

As discussed above, alarms may be provided for a number of desired conditions. For example, alarms or other alerts may be provided when a user's glucose level is approaching a predefined threshold, or has exceeded a predefined threshold, which may indicate that a user is approaching hypo- or hyper-glycemia. An alarm may be triggered by change in trends of analyte levels or by the current value of an analyte level. The alarm may be activated when a specific bolus amount is required to be dispensed. The alarm may indicate that an occlusion has occurred in a pump or that the syringe portion of a syringe-type infusion pump is not seated properly. The alarm may be an audio, visual, and/or tactile alarm. For an audible alarm, such as beeping, the alarm may get increasingly louder. For a tactile alarm, such as a vibration, the alarm may get increasingly stronger and/or faster. For a visual alarm, such as flashing or changing of color or indication of an alarm by an icon, the alarm may get increasingly brighter, faster, and/or larger. A visual alarm may also be conducted through SMS text messages on the monitor. In embodiments, the alarm may have a snooze option. In further embodiments, the alarm is through mp3's or system tones, such as beeping. In still further embodiments, the alarm is a personalized voice tag alarm, in which a parent, physician, caretaker, or other person may record a warning that plays upon activation (e.g. “your blood glucose is low,” “you need to take a bolus,” etc.).

An analyte sensor may feature this capability to track and reorder supplies. In one embodiment, the sensor has a capability to track how many lancets are used and to prompt the user to reorder lancets when a certain amount has been used. The sensor may also be programmed to automatically reorder lancets for the user by transmitting a message to the user's pharmacy or other predetermined supply source. The same feature could allow the user to input dosage amounts administered so that the sensor can track and account for the amount of insulin used, and reorder automatically when necessary. Instead of automatically reordering, the sensor may be configured to send a prompt or alert instead to the user to remind the user to reorder.

The sensor may include one or more alarms commonly known in the art, such as a reminder to inject or infuse insulin or to administer other medications. The sensor may have its alarms customized depending on whether the user administers the insulin through an infusion device or through an injection device. Users who administer multiple daily injections with an injection device may wish to group the dosages into larger amounts at time periods that are spread farther apart than infusion device users. In contrast to infusion device users, a MDI user has to inject himself or herself with a needle each time a dose is needed. Thus, it is more convenient to lump dosages together, when possible, and to administer the dosages at times farther apart. The sensor may be modified so that the alarm is spaced at farther intervals. The alarm may also have a “snooze” feature that allows the user to delay the alarm to a later time. This is particularly useful in cases where the user is preoccupied at the moment the alarm sounds, for example, driving a car, and needs to delay the alarm to remind the user to administer the dose at a later but more convenient time.

The alarms may be customized to specific user needs. The alarm may be set to flashing lights for the hearing impaired, or warning sounds and/or vibration for the vision impaired. There could further be included headphones that can plug into the analyte sensor for vision impaired to instruct the user on what to do in the case that an alarm goes off. The headphones could also be plugged into a MPEG player or the like.

In other embodiments, a speaker is included to provide an alternative mode of communication. In an embodiment, the analyte sensor, such as a BG sensor, may use the speaker to announce a message that states “move nearer to pump” when the sensor senses that the communication with the medication device is weak or interrupted. In the alternative, the analyte sensor may simply display a text message that states “move nearer to pump.” A similar message may be displayed if the BG sensor senses some type of problem or malfunction. Alternatively, an alarm may alert the user of any problem or malfunction by vibrating, emitting warning sounds, flashing light, and the like.

In further embodiments, the analyte sensor is adapted to receive additional information about a patient. For example, the analyte sensor may monitor heart rate or and/or metabolic rate, as in an exercise monitor. In further embodiments, the heart rate or metabolic rate may be correlated to a level of exercise, such as low, medium or high, to store in the analyte sensor memory, medication device memory, and/or other device memory. An analyte sensor, especially one that is worn on the skin, like a watch, may further be adapted to monitor the patient's temperature, salinity (from sweat), ketones, or other analyte characteristic. The analyte sensor may be adapted to measure further analyte characteristics, such as alcohol content of blood, as in a breathalyzer, ketones, and/or lactose. Further examples of analytes that may be monitored by the analyte sensor are discussed above.

The sounds of the analyte sensor may also be customizable, including, but not limited to sounds for alarms, key input, and alerts. Different audible features may be included in the module and/or may be downloaded from a computer.

Among other advantages, embodiments of the present invention may provide convenience and ease of use. For example, an embodiment with a user interface and display on the analyte sensor may cater to the active lifestyles of many insulin dependent diabetics. A large and simple display minimizes the potential for error in reading and interpreting test data. A small overall size permits discretion during self-monitoring and makes it easy to carry. In some embodiments, the sensor may include a dedicated backlight to facilitate viewing. The backlight may be a user programmable multi-color backlight that additionally performs the function of a visual indicator by flashing colors appropriate to the level of an alert or alarm. The backlight may also have variable intensity (automatic or manual) to preserve the battery power and improved viewing.

The power of the analyte sensor and of the other various devices discussed herein may be provided from a battery. The battery may be a single use or a rechargeable battery. Where the battery is rechargeable, there may be a connector or other interface on a device to attach the device to an electrical outlet, docking station, portable recharger, or so forth to recharge the battery while in the device. It is also possible that a rechargeable battery may be removable from the device for recharging outside of the device, however, in some cases, the rechargeable battery may be sealed into the housing of the device to create a more water resistant or waterproof housing. The devices may be adapted to accommodate various battery types and shapes. In embodiments, the devices may be adapted to accommodate more than one type of battery. For example, a device may be adapted to accommodate a rechargeable battery and, in the event of battery failure or other need, also adapted to accommodate a readily available battery, such as an AA battery, AAA battery, or coin cell battery.

In an embodiment of the present invention, the processor of the medication device uses power cycling such that power is periodically supplied to the communication system of the medication device until a communication is received from the sensor, for example, a BG sensor. When a communication is received from the sensor, the processor of the medication device discontinues using power cycling so that the power is continuously supplied to the medication device communication system. The medication device processor may then resume using power cycling upon completing the receipt of the communication including the data indicative of the determined concentration of the analyte in the user from the sensor communication system.

In further embodiments, the analyte sensor and its communication system are capable of being deactivated and reactivated. The sensor may include input devices, such as keys, buttons, and the like, for inputting commands, and the communication system of the sensor is capable of being deactivated in response to a first command from the user input device and being reactivated in response to a second command from the user input device. Alternatively, the communication system of the analyte sensor may be automatically reactivated after a predetermined amount of time has elapsed or at a predetermined time of day.

In embodiments, the sensor may be used to determine concentration of one of any variety of analyte types including, but not limited to, oxygen, blood, temperature, lactase, pH, and the like. In further alternative embodiments, the analyte sensor is a BG measurement sensor and may use samples from body fluids other than blood, such as interstitial fluid, spinal fluid, saliva, urine, tears, sweat, and the like. In still further embodiments, the analyte sensor may be utilized to determine the concentrations, levels, or quantities of other characteristics, analytes, or agents in the patient, such as hormones, cholesterol, oxygen, pH, lactate, heart rate, respiratory rate, medication concentrations, viral loads (e.g., HIV), or the like. In still other alternative embodiments, other fluids may be delivered to the user, such as medication other than insulin (e.g., HIV drugs, drugs to treat pulmonary hypertension, iron chelation drugs, pain medications, and anti-cancer treatments), chemicals, enzymes, antigens, hormones, vitamins, or the like. For pain management, a bolus function may be set up as a Patient Controlled Analgesic (PCA) function for customized delivery or the user may press a preset bolus button several times. Particular embodiments are directed towards the use in humans; however, in alternative embodiments, the infusion devices may be used in animals.

In other embodiments, where the analyte sensor is a BG sensor that determines BG level, the sensor may communicate the measurement of BG level to the medication device to determine the amount of insulin for delivery to the user. In alternative embodiments, the BG measurement sensor may be a continuous glucose measurement system, a hospital hemacue, an automated intermittent blood glucose measurement system, and the like, and/or the BG sensor may use other methods for measuring the user's BG level, such as a sensor in contact with a body fluid, an optical sensor, a RF sensor, an enzymatic sensor, a fluorescent sensor, a blood sample placed in a receptacle, or the like. The BG sensor may generally be of the type and/or include features disclosed in U.S. patent application Ser. No. 09/377,472 filed Aug. 19, 1999 and entitled “Telemetered Characteristic Monitor System and Method of Using the Same,” Ser. No. 09/334,996 filed Jun. 17, 1999 and entitled “Characteristic Monitor with a Characteristic Meter and Method of Using the Same,” Ser. No. 09/487,423 filed Jan. 20, 2000 and entitled “Handheld Personal Data Assistant (PDA) with a Medical Device and Method of Using the Same,” and Ser. No. 09/935,827 filed Aug. 23, 2001 and entitled “Handheld Personal Data Assistant (PDA) with a Medical Device and Method of Using the Same,” which are herein incorporated by reference. Such BG measure may be adapted to be carried by the user, for example, in the hand, on the body, in a clothing pocket, attached to clothing (e.g., using a clip, strap, adhesive, or fastener), and the like.

In some embodiments, the sensor may communicate to the medication device and other components via an intermediate controller device. In this embodiment, the controller device contains the electronic circuitry for intelligence. In further embodiments, the sensor may contain all or substantially all of the intelligence. In such an embodiment, the electronics will be contained in the sensor housing, and the sensor may communicate directly to the medication device and/or remote monitoring devices without an intermediate controller device. In embodiments, the different devices may include antennas to increase the receptivity available for transmission of information.

The amount of time that the sensor communicates with the medication device or other components may be limited to save power in the sensor. For example, radio-frequency (RF) communications may be minimized, such that the marriage between the medication device and sensor occurs once until further communication is necessary to exchange data. The information regarding the screens displayed is sent to the analyte sensor, and when the medication device needs to display a screen, it sends a screen number to the sensor. In the case of screen displays, if the data being sent is fixed, then the screen can be simply displayed. If the data is variable, then the variable data is sent with the screen to the medication device. Exchange IDs, strings to be displayed, and foreign languages are among data that may be sent from the analyte sensor. Further commands that may be sent from the medication device include, among other commands, a command to show a specific screen on the sensor, a command for displaying requested information on the screen, a command for showing the rules for the input devices, a command for showing the intelligence about that screen type (e.g., menus, data entries, etc.), and the like. The devices may all send diagnostic information to each other, and particularly to the sensor, so that the user may see if anything is going wrong with any of the devices.

Further examples include giving the analyte sensor cellular telephone, pager or watch capabilities. These embodiments integrate commonly used devices with the analyte sensor so that the user may have one less device to carry. For example, the sensor housing may include time-telling functions. For example, the sensor may be a wrist-worn device, such as a watch. The watch may include a credit card-sized display to facilitate easier viewing and adapted to display a time. The display of the time may be digital or analog. The time may be changed by the user using input devices like keys or buttons or a scroll wheel, depending on the set-up of the watch device. The watch display may be used to indicate the analyte levels, such as that of the user's glucose level. A watch having the above features is disclosed in U.S. patent application Ser. No. 11/496,606, entitled “Watch Controller for a Medical Device,” filed Jul. 31, 2006, which is hereby incorporated by reference in its entirety. The sensor may also be a watch that can be carried on other parts of the body or clothing, such as the ankle, neck (e.g., on a chain), pocket, or ankle. Other options for integrating with the sensor include but are not limited to a key fob, PDA's, smart phones, watch remotes, and the like. The analyte sensor may further communicate with, and download data such as software upgrades and diagnostic tools from, a remote station like a computer from a connector.

The BG sensor may communicate with a remote station, such as a computer, through a data transfer system, using a type of communication connector that couples the BG sensor to the computer and allows the data downloading. Alternatively, communication may be by wireless methods, such as RF, IR, Bluetooth or other wireless methods. Data may be downloaded via the RF telemetry in the same manner as data is transferred from the BG sensor to the medication device. The transmitter (or a transceiver) converts RF signals into compatible electrical pulses that may be subsequently sent through a serial port to a specified destination. Data, including software upgrades and diagnostic tools, may also be downloaded via RF telemetry, or any other wireless or wired method, from a remote station, such as the computer, to the medication device. Other remote stations include, but are not limited to, a hospital database, a cellular telephone, a PDA, a smart phone or internet. For example, a cellular phone may be used as a conduit for remote monitoring and programming. In one embodiment, the sensor may be configured so as to have cellular telephone capabilities. In further embodiments, the sensor and/or the other devices with display may be capable of providing PDA functions as well, removing the need for patients to carry separate PDA devices.

In specific embodiments, the BG analyte sensor includes a housing adapted to be carried by the user. A processor may be contained in the housing to process data and commands inputted by the user, and a transmitter (or a transceiver) contained in the housing and coupled to the processor transmits such communications, including data indicative of the determined concentration of the BG in the user, to a medication device, such as an infusion medication device or a single dose medication device. In further embodiments, the electronics may be integrated with the BG sensor in one housing.

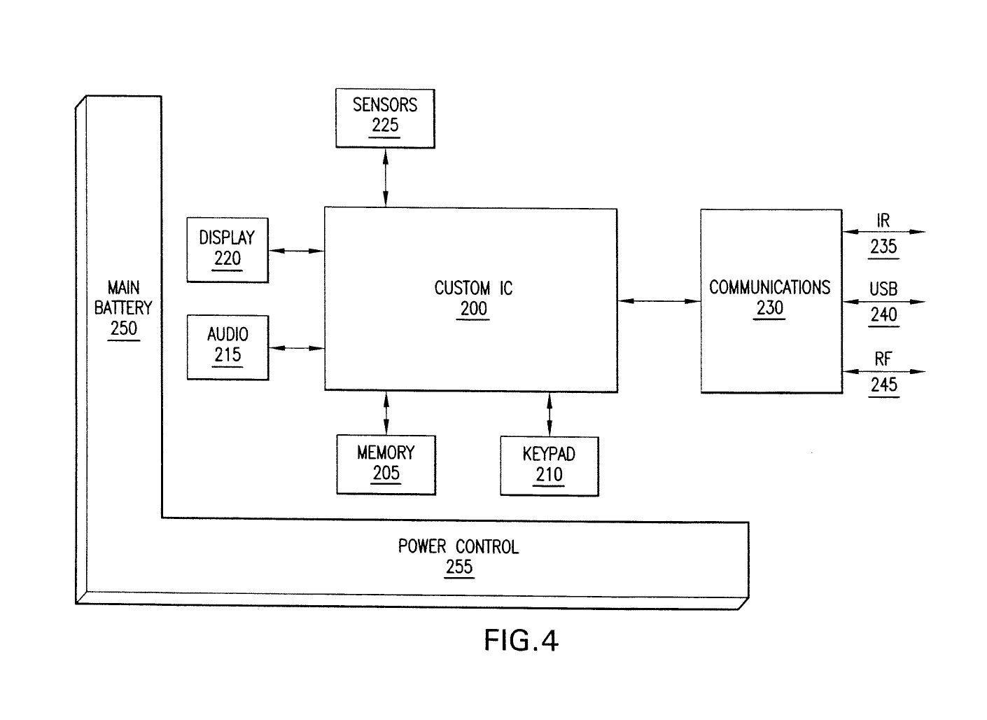

FIG. 4 shows an electronics architecture according to an embodiment of the invention with a custom integrated circuit (“custom IC”) 200 as the electronics processor. This architecture can support many of the devices discussed herein, for example the analyte sensor, the medication device, the controller device, or any combination of the above. The custom IC 200 is in communication with a memory 205, keypad 210, audio devices 215 (such as speakers or audio electronic circuitry such as voice recognition, synthesis or other audio reproduction), and a monitor or display 220. The custom IC 200 is in communication with the sensor 225 included in the device, or in communication with the device (for example, a BG sensor or a device which includes an analyte determining function). The electronics architecture further may include a communications block 230 in communication with the custom IC 200. The communications block 230 may be adapted to provide communication via one or more communications methods, such as RF 235, a USB 240, and IR 245. In further embodiments, the custom IC 200 may be replaced by electronic circuitry, discrete or other circuitry, with similar functions.

The electronics architecture may include a main battery 250 and a power control 255. The power control 255 may be adapted to give an end of battery warning to the user, which can be predicted based on the type of battery used or can be calculated from the power degradation of the battery being used. However, in certain embodiments it is not necessary to know the type of battery used to create an end of battery warning. Various battery types, such as rechargeable, lithium, alkaline, etc., can be accommodated by this design. In certain embodiments, the electronics architecture includes a removable battery and an internal backup battery. Whenever a new removable batter is inserted, the internal backup battery will be charged to full capacity and then disconnected. After the removable battery has been drained of most of its energy, it will be switched out of the circuit and the internal backup battery will be used to supply power to the device. A low battery warning may then be issued. The internal backup battery may be rechargeable. In further embodiments, a supercap, for example, is used to handle the peak loads that the rechargeable internal battery could not handle directly, because it has sufficient energy storage. This method also allows the use of any type of removable battery (alkaline, lithium, rechargeable, etc.) and partially drained batteries. Depending on use, the backup battery may allow the device to operate for at least one day after the removable battery has been drained or removed. In further embodiments, a microprocessor measures the charge states and control switches for removable and internal backup batteries.

The analyte sensor may also include expanded capabilities, such as for example, voice synthesis, voice activation, polyphonic speakers for the vision impaired, and plugs on the sensor for headphones. Likewise, a controller device may also be configured to provide these expanded capabilities.

The analyte sensor may also talk directly to an optional peripheral devices that include a physiological characteristic sensor, such as a telemetered glucose monitoring system (TGMS) sensor. The TGMS sensor is inserted into the subcutaneous tissue of the user to read body fluids, and allows for continuous blood glucose monitoring. The readings are used in conjunction with the BG level determined by the analyte sensor to continuously monitor BG levels through extrapolating the BG measurements. This embodiment would be compatible with users that do not have a medication device, in which case, there is a need for the ability to talk directly to the TGMS sensor without talking to the medication device.

If the BG sensor talks to the TGMS sensor, then the TGMS sensor may broadcast the data received from the BG sensor to the medication device, such as an infusion pump device. In some embodiments, the system is set up to automatically call for assistance when analytes reach a certain level. The system may also include a global positioning system (GPS), such as ONSTAR (sold by OnStar Corp.), to provide a more efficient manner with which to locate the user. GPS functions may be included separately from cellular telephone type functions.

In an embodiment of the present invention, the graph displayed on the analyte sensor may display information regarding boluses, finger sticks, exercise, meals and the like. In one embodiment, the graph displayed has eight segments, representing different limits and an actual BG line. In other embodiments, the graphs may include additional time spans for which to show the varying BG levels. For example, the embodiments may include a 3, 6, 12, and 24 hour graphs. Additional features of the graphs may include the ability to zoom in or out of the graph. There may be included an ESC key that will allow the user to return to the last scale. Other options may allow the user to focus on specific positions on a graph. In yet another feature, the user can select the resolution in which to view the graph.

In embodiments, the analyte sensor includes a “bolus estimator” program which allows the sensor to take into account a variety of factors that may affect blood glucose levels of the user which may in turn affect the amount of insulin needed. For example, in one embodiment, the bolus estimator factors in the other medications that the user is ingesting, especially those that will affect glucose sensitivity, such as for example, glucophage. In other embodiments, the bolus estimator will enable the sensor to factor into the insulin dosage what device the insulin is to be administered through because different devices will administer medication differently. Factoring this differential into the dosage is especially important for those patients who use multiple daily injections rather than infusion devices, as their dosages may change depending on the device they select to inject the insulin.

In further embodiments, the sensor may include capabilities such as setting insulin sensitivity and insulin/carbohydrate ratios. This capability allows users to customize settings of the sensor. For example, the bolus estimator may come with educational tools and protocols that will allow a user to set their insulin sensitivity by ingesting specific foods in specific amounts and analyzing how their blood glucose level fluctuates and/or responds to specific amounts of insulin administered. The results from the analysis can be stored into the sensor memory to apply to the user's settings. In addition, the sensor may also store in memory a database of medications, for example, those that affect insulin sensitivity for future reference. This data may be programmed into the sensor and/or downloaded from specific internet sites. The sensor may also be programmed to prompt alerts to the user when a medication that may affect insulin sensitivity is ingested.

The sensor may also have other user prompts. In one embodiment, the sensor prompts the user to report events that help create event markers that can further help gauge the user's sensitivity to various factors. If there is a rapid increase or decrease in blood glucose level, the sensor realizes the change and will prompt the user with a text message or audio message asking “what just happened—did you just exercise?,” “did you just eat?,” “input what you just ate,” and the like. The information inputted by the user will allow the sensor to analyze how the blood glucose level fluctuates or reacts to specific events. Cataloging such events can help user note, for example, how fast insulin or other medications affect blood glucose level or how much certain foods affect blood glucose level. These events may include, but are not limited to, type of food ingested, amount of food ingested, amount of exercise undertaken, type of drug ingested, amount of drug ingested, type of medication device used, time lapse from last bolus administered, and user sensitivity. Recording specific events may allow a physician or caretaker better monitor and manage the patient's diet and dosage schedules. This information may also be communicated to and monitored through a data management software program like CARELINK (sold by Medtronic Minimed, Inc.). Furthermore, the sensor may be able to organize the sensitivity and/or response patterns from these external factors into a chart for easier analysis and calculation of bolus amount.

In embodiments used with data management software, the sensor may undergo periodic uploads of data, for example, in the middle of the night. These uploads may be performed automatically, without any action on the part of the user. The uploads may include data to upgrade or update the sensor from the central data management station. The uploads may also include data sent by a physician or caretaker via a computer network. Alternatively, the uploads may be conducted via a wire connected between the sensor and the source of the uploaded data. The data management software, such as CARELINK, may also incorporate a SMS server so that messages may be delivered in the form of text messages, as in cellular telephones. The sensors may be adapted to recognize whenever they are in the presence of a management station and upload all the data that those sensors do not already have and save the data to a repository.

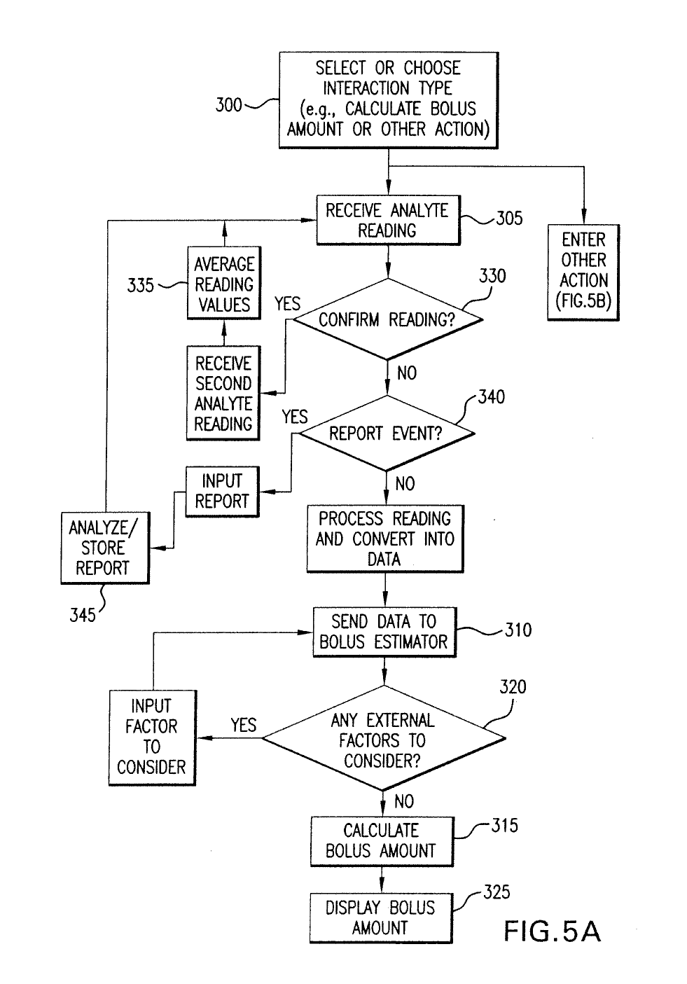

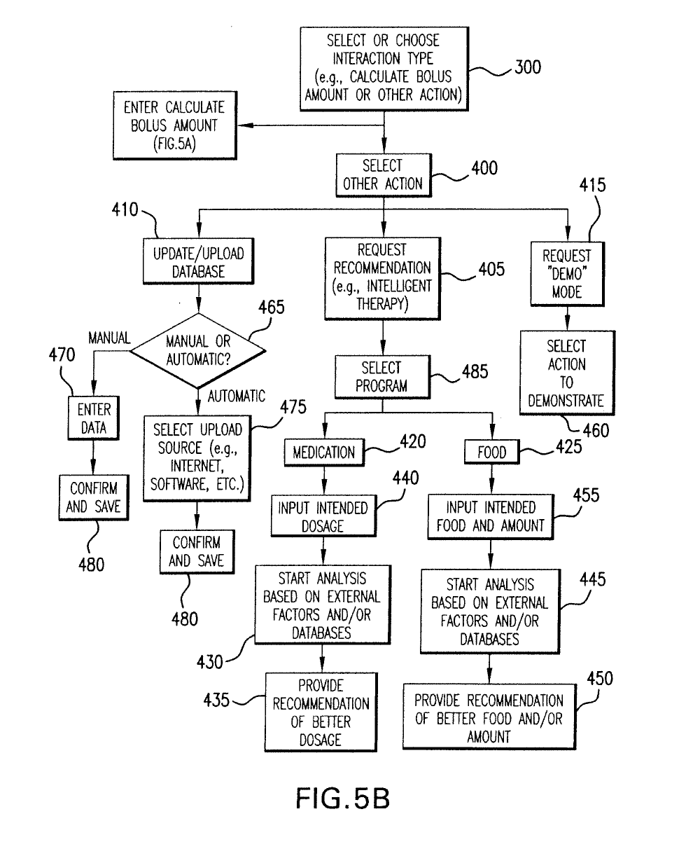

In further embodiments, as shown in FIGS. 5A and 5B, the analyte sensor may include various menu options to provide an accurate bolus amount or “intelligent therapy,” in which the sensor is able to analyze and make suggestions for dosage amounts and dosage schedules that better fit the user's profile based on various factors such as insulin sensitivity, analyte patterns and the like.

Embodiments, shown in FIG. 5A, provide a method of diabetes management that is involves a selection of actions 300. The method for calculating a bolus amount involves receiving a plurality of readings 305 over time, either automatically or manually, from an analyte sensor, processing each of the readings to generate data 310, using the data to calculate a bolus amount 315 of medication as a function of the data in combination with external factors 320, and directing a user to deliver the bolus amount 325. The bolus amount is delivered through a single dose medication device in particular embodiments. An external factor is one that can affect the calculation of the bolus amount, such as for example, meal consumption, exercise, medication intake, time lapse from the last bolus dispensed, type of medication device used and user sensitivity. The receiving step may involve obtaining two or more actual analyte level readings 330 (to confirm the value used), of which an average is taken 335, to yield the final reading. In further embodiments, the directing step is performed by displaying the bolus amount on a monitor on the analyte sensor.

These embodiments may also remember the user's profile and schedule so that the sensor can prompt or alert the user to take some action if the user forgets. For example, the sensor may remind the user to report 340 whether food was consumed or exercise was conducted or the sensor may alert the user if a dose of insulin was missed. The report is stored in databases 345 that can be referenced to in analyzing and calculating bolus amounts.

As shown in FIG. 5B, other actions may also be selected 400. Some of these embodiments include requesting “intelligent therapy,” in which the sensor is able to analyze and make suggestions for dosage amounts and dosage schedules or food types and amounts that better fit the user's profile 405, updating or uploading existing databases 410, and requesting a demonstration on using the sensor device 415. The user may select from a variety of programs to demonstrate usage 460, such as how to use intelligent therapy.

By requesting intelligent therapy 405, the user may select the program desired 485, such as for example, the medication recommendation 420 or the food recommendation 425 option. In the medication recommendation option 420, the sensor device can analyze the current analyte level against a background of external factors such as user history and sensitivity 430. This analysis can also take into account information stored in the various databases. From the analysis, the sensor device may suggest a dosage amount 435. In some cases, the user may enter an intended dosage amount 440 and request that the sensor perform the analysis to suggest a better dosage amount 435.

In the food recommendation option 425, the sensor device can analyze the current analyte level against a background of external factors such as user history and sensitivity 445 and also take into account information stored in the various databases to provide a suggestion for food intake 450. From the analysis, the sensor device may what foods, and in what amounts, should be consumed. In some cases, the user may enter an intended meal consumption 455 and request that the sensor perform the analysis to suggest a better meal to intake 450.

By selecting the update/upload option 410, the user may specify when and how information is entered into the sensor device memory and stored. For example, the user may choose manually or automatically to enter data into the memory 465. If there is specific piece of information, the user may use the inputs to manually enter the information 470. The user may also direct the sensor device to automatically upload information from a source at regular or periodic intervals, for example, nighttime hours. The upload source may be from, for example, a software program, a computer, or the Internet 475. An confirmation prompt 480 may be included to ensure that the correct and desired information is being saved.

Additional steps for the diabetes management method may involve automatically tracking the amounts of various diabetes supplies used. For example, the user may input the total amount of lancets, insulin or insulin syringes purchased. The analyte sensor device may then be able to count the number of lancets used or amount of insulin dispensed, and subtract the amount used from the total amount. When supplies run low, the analyte sensor may sound an alarm to alert the user to this fact. There may also be included a step in which an automatic reorder for the supply may be sent.

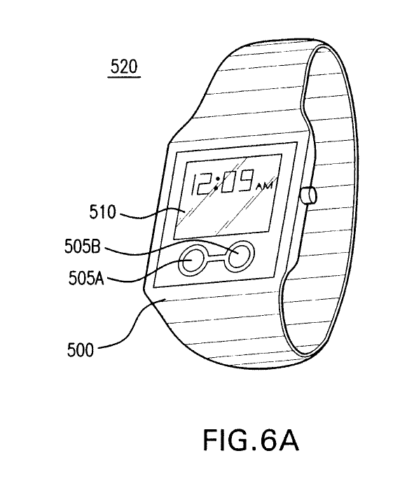

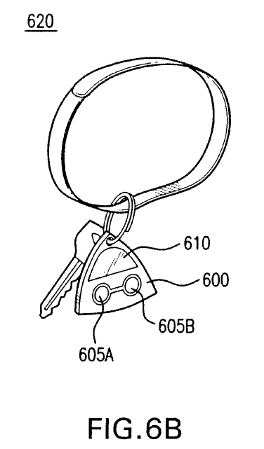

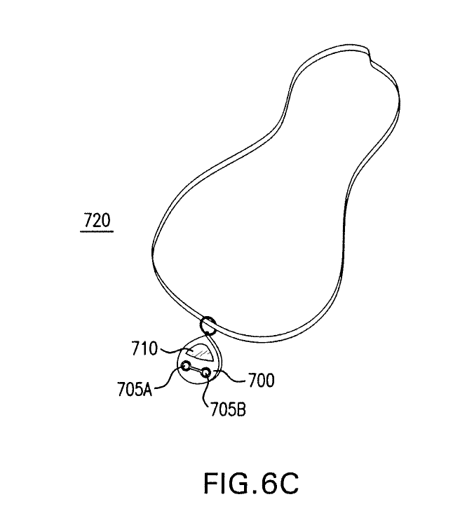

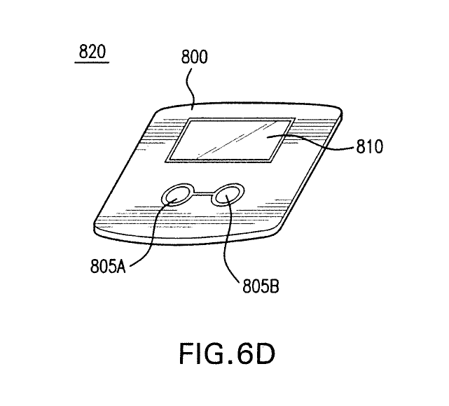

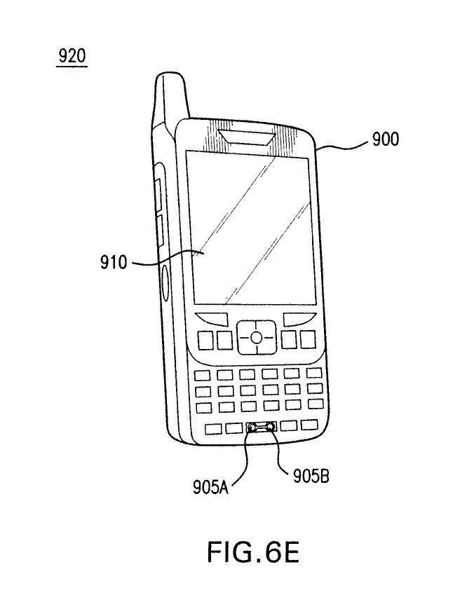

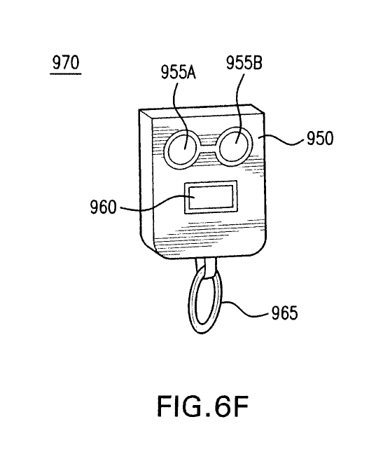

In various embodiments, a sensor may be integrated with a display or monitor so that less equipment is necessary for the user to handle. As shown in FIG. 6A, the sensor housing 500 may also be a watch. In this manner, the sensor device 520 can be carried on other parts of the body or clothing, such as the ankle, neck (e.g., on a chain), pocket, or ankle. The watch housing 500 may include one or more inputs 505A and 505B, and a monitor 510 on which to display the time as well as the bolus amount or other related information.

In other embodiments, shown in FIG. 6B, the sensor housing 600 may also be a keychain accessory. In this manner, the sensor device 620 can be carried easily by the user on a keychain with the user's other keys. The keychain accessory housing 600 may include one or more inputs 605A and 605B, and a monitor 610 on which to display the bolus amount or other related information.

In FIG. 6C, the sensor housing 700 may also be a charm that attaches to a user's jewelry, such as a bracelet or necklace (as shown). In this manner, the sensor device 720 can be carried on other parts of the body conveniently. The watch housing 700 may include one or more inputs 705A and 705B, and a monitor 710 on which to display the bolus amount or other related information.

In FIG. 6D, the sensor housing 800 may be integrated into an accessory card. In this manner, the sensor device 820 can be carried conveniently in the user's clothing, purse, or wallet. The accessory card housing 800 may include one or more inputs 805A and 805B, and a monitor 810 on which to display the bolus amount or other related information.

In some embodiments, shown in FIG. 6E, the sensor housing 900 may also be integrated into a Smartphone. In this manner, the sensor device 920 can be carried as part of the user's phone and to reduce the number of accessories that the user needs to carry. The Smartphone housing 900 may include one or more inputs 905A and 905B for use with the sensor device 920, and a monitor 910 on which to display the Smartphone interfaces as well as the bolus amount or other related information.

In yet other embodiments, as shown in FIG. 6F, the sensor housing 950 may also be a key fob. In this manner, the sensor device 970 can be carried easily by the user in clothing or in an accessory such as a purse. The key fob housing 950 may include one or more inputs 955A and 955B, and a monitor 960 on which to display the bolus amount or other related information. A hand strap 965 may be included with the sensor device 970 for further convenience.

Some embodiments may include a barcode reader in the sensor which will allow the sensor to recognize different food items by the barcode on the packaging. The sensor may recognize the food item and automatically input the carbohydrate information into the user's schedule information and count the carbohydrates in calculating insulin dosage to be delivered. The barcode readings can be compiled into a barcode library for easy reference. The barcode library may be a database built directly into the sensor memory or the data may be downloaded from websites that list the information correlating to the specific barcodes. Other databases that may be compiled or downloaded include a food library that stores information of the amount of carbohydrates or other nutritional values correlating to each food item, a user history that stores information regarding the user's daily schedules, patterns or sensitivities, and a drug library that stores information about various drugs that may be taken and how each drug affects insulin intake.

In addition to the Internet, the databases may be downloaded through a transceiver embodied by the user's cellular telephone. Downloads may also be conducted automatically by the sensor device during specific times, such as for example, nighttime hours. Other options may include eliminating the need to bypass the transceiver every time a food item is selected, such as, downloading the food items from a PC or software and storing it until use. The user may also manually input the information. The websites may also be used to post automatic updates to the barcode information so that the information is kept up to date. Variable data could be included for a small food library with less than 50 food items. For example, there could be variable data for a food library dedicated to breakfast foods only. There could be a “breakfast” key or icon on the sensor that the user can select. There may also be “lunch” and “dinner” and “snack” icons. The carbohydrate counting books and/or food libraries may also be downloaded from sources such as a website. The sensor may have the capability of serving as a nutritionist, advising the user on how to improve his or her diet or suggest better foods to select.

In embodiments, the sensor may include other additional features that make the sensor more convenient to use. For example, some embodiments have a “demo” mode in which the sensor may provide a demonstration of how different functions work to the user. Other embodiments have voice tags with which the alerts or audio instructions will be played. These voice tags will allow the user to record the audio with a specific voice, such as that of a parent or caretaker, so that alerts or instructions are played with that voice. Embodiments may also have a pedometer integrated into the sensor that can track exercise, whether it is at a high level or a low level. The user may have the option to input the data that the pedometer collects to help determine appropriate insulin dosages. In other embodiments, the sensor can be instructed to calibrate itself when the blood glucose readings are stabilized. In still other embodiments, the sensor may be calibrated using data from the medication device.

The sensor may also have an accessory card reader that can register food information into the glucose sensor to determine whether a food item is recommended for a meal. Such an accessory may be used by parents or caretakers as a debit card with which children can purchase meals at a school cafeteria. Only food items that are approved by the parent or caretaker will be so recognized by the card reader and be purchasable by the card.

In embodiments, the sensor may also include a basal estimator which helps to take the information generated by the user and/or bolus estimator and calculates the user's basal flow rate and determines the impact, if any, on the insulin dosages. The basal estimator may provide other features such as suggesting how to better use lancets, and other equipment.

In yet another embodiment, the analyte sensor may communicate with a bedside monitor. The monitor could communicate through the same avenues as described above with the other peripheral devices. The monitor could be used, as described above, to remotely alarm people other than the user, such as for example, parents, physicians, nurses, and the like. This would provide an extra layer of monitoring for the user, especially when the user is alone. In further embodiments, the system may be set up so that multiple devices are placed around the house. This would provide easy access to monitor the diabetic. Additionally, the parent will be able to obtain data to monitor a child user at home and when the parent is away. Such home monitors could be set to any mode preferred, for example, flashing lights, warning sounds like beeping, vibration, and the like. There may further be included a turn-off option where, if there is not a need to communicate with the sensor, the user can choose a selection to turn off the sensor. In further embodiments, there may be included a feature in any of the devices including an alarm where when the device has sounded an alarm for a period of time and the user has not responded, the alarm will switch to a vibrate mode and/or will attempt to signal companion devices in the system to alarm the user. Other features may include a function that allows the remote user (parent, physician, nurse, etc.) to change and/or deliver a bolus from remote sites using the analyte sensor.

The cellular network could provide a conduit for remote monitoring and programming. Additionally, the cellular network could be used to notify parents, physicians, or emergency services of alarms or alert states. For example, the analyte sensor system may be set up to automatically call for assistance when analytes reach a certain level. A button may be included on the analyte sensor to automatically alert a parent, physician, or emergency services when pressed. For example, a monitoring device may be built directly into a patient's cellular telephone so that in the case of a hypoglycemic event, an alarm or connection may be made to emergency services via the cellular telephone. In a further embodiment, global positioning system (GPS) technology may also be built into the cellular telephone to allow easy location of the patient. Alternatively, GPS technology may be included in the sensor without cellular telephone technology. In other embodiments, the GPS technology may also be built into other devices used with the sensor.

It is noted that some users can be expected to have somewhat diminished visual and tactile abilities due to the complications from diabetes or other conditions. Thus, the display and buttons or other input devices may be configured and adapted to the needs of a user with diminished visual and tactile abilities. In alternative embodiments, the analyte sensor and/or associated devices may communicate to the user by audio signals, such as beeps, speech or the like.

Other display settings may be customizable, including, but not limited to, the background, sounds, fonts, color schema and wallpaper. The complexity of the interface may be customized to the sophistication of the user. For example, there may be an expert mode or a regular mode. Further, there may be a children's mode, with limited features available so that a child cannot dispense too much medication at once. Different display features may be included in the module and/or may be downloaded from a computer. The analyte sensor may have a memory with which to store customized settings or medication delivery control. The memory may be of any type that is known in the art, such as a volatile or non-volatile memory. Both a volatile and non-volatile memory may be used, which can speed up operation of the medication device. As an example, non-volatile memories that could be used in the invention include flash memories, thumb drives and/or memory sticks such as USB thumb drives, removable hard drives, and optical drives.

In further embodiments, the analyte sensor is made to be waterproof so that the function is not impaired should the sensor inadvertently come into contact with water. The analyte sensor may also be made of specific materials that give the sensor improved impact resistance to prevent chipping or shattering of the sensor if the sensor is dropped or otherwise impacted.

In some embodiments, the language that the analyte sensor operates in may comprise several different languages, ranging from 1 language to about 40 languages and potentially more. To set language, data must be first initialized to modify the phrases and detail font that may be significantly different in one language as compared to another language. For example, some languages, such as Chinese, are read in vertical columns, from the right to the left, and thus, needs to be displayed in such manner. One way to overcome this complication in using different languages is to have fonts built into the sensor. Because fonts are now described in pen strokes (true-type fonts), rather than in pixels (bit-by-bit) this allows the sensor to determine out how to display the different fonts. Another option could involve uploading the fonts in strings from various sources, such as the internet.

According to yet another embodiment of the present invention, a medication delivery system includes an analyte sensor, with a sensor display, and a medication device, and a method for delivering a fluid into a body of a user is provided. The method includes the steps of: receiving data communication from a user, transmitting with the analyte sensor the communication including data to a medication device, receiving with the medication device the communication, and displaying with the analyte sensor display information regarding the fluid delivery, where the display on the analyte sensor shows information according to instructions or communications sent to the sensor from the medication device. In embodiments, the display of the medication device may correspond with what is displayed on the sensor device display at any moment. The method may further include the step of displaying trends and graphs.

Although the above description has been focused on use of an analyte sensor with a medication device, it is appreciated that an analyte sensor as described herein could be used with any number of therapy/diagnostic devices. For example, in any case where a therapy/diagnostic device is tethered to the body, at least partially implanted in the body, or otherwise inconvenient for the user to manipulate while therapy or diagnosis is being performed, an analyte sensor may be used that can send commands to the therapy/diagnosis device and/or mimic the display on the therapy/diagnosis device. Therapies other than delivery or infusion of fluids could include electrical therapy, such as electrical therapy for the brain and for conditions such as epilepsy. Diagnostics could include any number of diagnostics, such as information from cardiac and other sensors.

Electrical therapy devices include neurostimulation devices for epilepsy, similar devices for pain management, etc. In addition, there are electro-acupuncture devices, where a needle is inserted into the body much like acupuncture, but additional therapy is delivered by electrical impulses. In certain embodiments, the structure of an electrical therapy device may include a needle that is inserted into appropriate areas of the body. The architecture would be similar to that of the devices described above. The patient/user would use the sensor to sense and alleviate pain and manage neurological symptoms on demand such as twitching, uncontrolled movement of limbs, spasms, and so forth by sending instructions to a medication device to deliver appropriate “dosages” of electrical impulses.

In further embodiments the sensor may include a medical alert display on the display or a medical alert on another part of the housing, to indicate a condition, such as an allergy or disease that should be alerted to medical professionals and others who may have to care for the user.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A sensor device for producing data indicative of an analyte level of a user, the sensor device comprising:
a sensor that measures an analyte level of a user;
sensor electronics coupled to the sensor that receive and process the measured analyte level to generate analyte data;
a bolus estimator that receives the analyte data from the sensor electronics and estimates a bolus amount of medication to be dispensed from a single dose medication device based on the analyte data and information about an intended meal planned to be consumed by the user, wherein the bolus estimator includes a memory that stores one or more databases used in estimating the bolus amount; and
a monitor coupled to the bolus estimator that displays a user interface, the monitor having one or more inputs that are used to enter and receive said information about the intended meal, and wherein the user interface displays the estimated bolus amount, wherein:
the sensor device prompts the user to report events when significant changes in the analyte level are sensed, said events being selected from the group consisting of exercise, medication intake, and type of medication device used;
the sensor device calculates user sensitivity based on the reported events;
the bolus estimator estimates the bolus amount based on the calculated user sensitivity; and
based on said analyte data, said information about the intended meal, and said one or more databases, the bolus estimator provides a recommendation of a suggested meal, said suggested meal being different from said intended meal.

2. The sensor device of claim 1, wherein the one or more databases are selected from the group consisting of a user history, a food library, a drug library, and a bar code library.

3. The sensor device of claim 2, wherein said recommendation of the suggested meal is based on an analysis of the user history.

4. The sensor device of claim 2, wherein said recommendation of the suggested meal includes a recommendation of food type and a recommendation of food amount to be consumed by the user.

5. The sensor device of claim 2, wherein the bolus estimator further provides a recommendation of medication dosage amount and medication dosage timing based on an analysis of the user history.

6. The sensor device of claim 1, wherein the sensor device conducts carbohydrate counting based on the one or more databases.

7. The sensor device of claim 1, wherein the one or more databases are updated from a source selected from the group consisting of software, Internet, and manual input.

8. The sensor device of claim 7, wherein the update takes place during nighttime hours.

9. The sensor device of claim 1, wherein the sensor continuously measures the analyte level of the user.

10. The sensor device of claim 1, wherein the sensor is subcutaneous.

11. The sensor device of claim 1, further including an indication device that provides at least one indication that the bolus amount to be dispensed has been calculated, wherein the at least one indication is selected from the group consisting of a visual indication, an audible indication, and a tactile indication.

12. The sensor device of claim 1, wherein the single dose medication device is selected from the group consisting of an inhaler, a jet injector, an injection pen, and a syringe.

13. The sensor device of claim 1, wherein the single dose medication device is disposable.

14. The sensor device of claim 1, wherein the one or more inputs are selected from the group consisting of buttons, keys, tabs, push pads, touch screens, and turn dials.

15. The sensor device of claim 1, further including a transmitter device coupled to the bolus estimator, wherein the transmitter device wirelessly transmits the bolus amount to the single dose medication device.

16. The sensor device of claim 15, further including an antenna attached to the transmitter device for increasing reception.

17. The sensor device of claim 15, wherein the wireless transmission is selected from the group consisting of radio frequency, infrared, and WiFi.

18. The sensor device of claim 15, wherein the wireless transmission is selected from single frequency communication, spread spectrum communication, Listen Before Talk (LBT), and frequency hopping communication.

19. The sensor device of claim 15, wherein the transmitter device transmits a communication to data management software.

20. The sensor device of claim 1, wherein the analyte level being measured is blood glucose level.

21. The sensor device of claim 20, wherein the sensor measures the blood glucose level after the blood glucose level is stabilized.

22. The sensor device of claim 1, wherein the medication is insulin.

23. The sensor device of claim 1, wherein the user interface presents data in graphical depictions.

24. The sensor device of claim 23, wherein the graphical depiction is selected from the group consisting of a graph, a chart, a extrapolation, a pie chart, and a table.

25. The sensor device of claim 23, wherein the user interface has a user-interactive demonstrative mode.

26. The sensor device of claim 1, further including a pedometer coupled to the bolus estimator, wherein the pedometer tracks the user's exercise and is used in conjunction with the bolus estimator to calculate the bolus amount.

27. The sensor device of claim 1, further including at least one alarm selected from the group consisting of a visual alarm, an audible alarm, and a tactile alarm.

28. The sensor device of claim 27, wherein the alarm activates when the analyte level of the user meets a predetermined threshold.

29. The sensor device of claim 27, wherein the alarm activates when a specific bolus amount is required.

30. The sensor device of claim 27, wherein the alarm grows in intensity.

31. The sensor device of claim 27, wherein the alarm includes a snooze option.

32. The sensor device of claim 27, wherein the visual alarm is sent through SMS text messaging.

33. The sensor device of claim 27, wherein the audible alarm is selected from the group consisting of beeping, voice tags, and MP3s.

34. The sensor device of claim 27, wherein the tactile alarm is sent through vibrations.

35. The sensor device of claim 1, further including a housing to contain the bolus estimator and the monitor.

36. The sensor device of claim 35, wherein the housing is selected from the group consisting of a keychain, a watch, a piece of jewelry, an accessory card, a Smartphone, and a key fob.

37. The sensor device of claim 35, further including a receptacle formed in the housing, wherein the receptacle receives a fluid from the user, and wherein the sensor electronics measure the analyte level of the user from the fluid.

38. The sensor device of claim 37, wherein the fluid is received into the receptacle on a test strip.

39. A sensor device for producing data indicative of an analyte level of a user, the sensor device comprising:
a sensor that measures an analyte level of a user;
sensor electronics coupled to the sensor that receive and process the measured analyte level to generate analyte data;
a bolus estimator that receives the analyte data from the sensor electronics and estimates a bolus amount of medication to be dispensed from a single dose medication device based upon the analyte data in combination with external factors; and
a monitor coupled to the bolus estimator that displays a user interface, the monitor having one or more inputs that are used to enter and receive information about the external factors, and wherein the user interface displays the estimated bolus amount,
wherein the sensor device prompts the user to report events when significant changes in the analyte level are sensed, said events being selected from the group consisting of meal consumption, exercise, medication intake, and type of medication device used, wherein the sensor device calculates user sensitivity based on the reported events, and wherein the bolus amount is estimated based on the calculated user sensitivity.

40. The sensor device of claim 39, wherein the sensor continuously measures the analyte level of the user.

41. The sensor device of claim 39, wherein the sensor is subcutaneous.

42. The sensor device of claim 39, further including an indication device that provides at least one indication that the bolus amount to be dispensed has been calculated, wherein the at least one indication is selected from the group consisting of a visual indication, an audible indication, and a tactile indication.

43. The sensor device of claim 39, wherein the single dose medication device is selected from the group consisting of an inhaler, a jet injector, an injection pen, and a syringe.

44. The sensor device of claim 39, wherein the single dose medication device is disposable.

45. The sensor device of claim 39, wherein the one or more inputs are selected from the group consisting of buttons, keys, tabs, push pads, touch screens, and turn dials.

46. The sensor device of claim 39, further including a transmitter device coupled to the bolus estimator, wherein the transmitter device wirelessly transmits the bolus amount to the single dose medication device.

47. The sensor device of claim 46, further including an antenna attached to the transmitter device for increasing reception.

48. The sensor device of claim 46, wherein the wireless transmission is selected from the group consisting of radio frequency, infrared, and WiFi.

49. The sensor device of claim 46, wherein the wireless transmission is selected from single frequency communication, spread spectrum communication, Listen Before Talk (LBT), and frequency hopping communication.

50. The sensor device of claim 46, wherein the transmitter device transmits a communication to data management software.

51. The sensor device of claim 39, wherein the external factors are selected from the group consisting of meal consumption, exercise, medication intake, time lapse from last bolus dispensed, type of medication device used, and user sensitivity.

52. The sensor device of claim 39, wherein the analyte level being measured is blood glucose level.

53. The sensor device of claim 52, wherein the sensor measures the blood glucose level after the blood glucose level is stabilized.

54. The sensor device of claim 39, wherein the medication is insulin.

55. The sensor device of claim 39, wherein the user interface presents data in graphical depictions.

56. The sensor device of claim 55, wherein the graphical depiction is selected from the group consisting of a graph, a chart, a extrapolation, a pie chart, and a table.

57. The sensor device of claim 55, wherein the user interface has a user-interactive demonstrative mode.

58. The sensor device of claim 39, further including a pedometer coupled to the bolus estimator, wherein the pedometer tracks the user's exercise and is used in conjunction with the bolus estimator to calculate the bolus amount.

59. The sensor device of claim 39, further including at least one alarm selected from the group consisting of a visual alarm, an audible alarm, and a tactile alarm.

60. The sensor device of claim 59, wherein the alarm activates when the analyte level of the user meets a predetermined threshold.

61. The sensor device of claim 59, wherein the alarm activates when a specific bolus amount is required.

62. The sensor device of claim 59, wherein the alarm grows in intensity.

63. The sensor device of claim 59, wherein the alarm includes a snooze option.

64. The sensor device of claim 59, wherein the visual alarm is sent through SMS text messaging.

65. The sensor device of claim 59, wherein the audible alarm is selected from the group consisting of beeping, voice tags, and MP3s.

66. The sensor device of claim 59, wherein the tactile alarm is sent through vibrations.

67. The sensor device of claim 39, wherein the bolus estimator includes a memory to store information.

68. The sensor device of claim 67, wherein the memory stores one or more databases used in estimating the bolus amount.

69. The sensor device of claim 68, wherein the one or more databases are selected from the group consisting of a user history, a food library, a drug library, and a bar code library.

70. The sensor device of claim 69, wherein the bolus estimator provides intelligent therapy to the user based on the one or more databases.

71. The sensor device of claim 70, wherein the intelligent therapy comprises a recommendation of medication dosage amount and medication dosage timing based on an analysis of the user history.

72. The sensor device of claim 68, wherein the sensor device conducts carbohydrate counting based on the one or more databases.

73. The sensor device of claim 68, wherein the one or more databases are updated from a source selected from the group consisting of software, Internet, and manual input.

74. The sensor device of claim 73, wherein the update takes place during nighttime hours.

75. The sensor device of claim 39, further including a housing to contain the bolus estimator and the monitor.

76. The sensor device of claim 75, wherein the housing is selected from the group consisting of a keychain, a watch, a piece of jewelry, an accessory card, a Smartphone, and a key fob.

77. The sensor device of claim 75, further including a receptacle formed in the housing, wherein the receptacle receives a fluid from the user, and wherein the sensor electronics measures the analyte level of the user from the fluid.

78. The sensor device of claim 77, wherein the fluid is received into the receptacle on a test strip.

79. A sensor device for producing data indicative of an analyte level of a user, the sensor device comprising:
a sensor that measures an analyte level of a user;
sensor electronics coupled to the sensor that receive and process the measured analyte level to generate analyte data;
a first transmitter device coupled to the sensor electronics, wherein the first transmitter device wirelessly transmits a communication including the analyte data;
a bolus estimator that receives the communication from the first transmitter device and estimates a bolus amount of medication to be dispensed from a single dose medication device based upon the analyte data in combination with external factors; and
a monitor coupled to the bolus estimator that displays a user interface, the monitor having one or more inputs that are used to enter and receive information about the external factors, and wherein the user interface displays the estimated bolus amount,
wherein the sensor device prompts the user to report events when significant changes in the analyte level are sensed, said events being selected from the group consisting of meal consumption, exercise, medication intake, and type of medication device used, wherein the sensor device calculates user sensitivity based on the reported events, and wherein the bolus amount is estimated based on the calculated user sensitivity.

80. The sensor device of claim 79, further including a second transmitter device coupled to the bolus estimator, wherein the second transmitter device wirelessly transmits the bolus amount to the single dose medication device.

81. The sensor device of claim 79, wherein the sensor is subcutaneous.

82. The sensor device of claim 79, wherein the external factors are selected from the group consisting of meal consumption, exercise, medication intake, time lapse from last bolus dispensed, type of medication device used, and user sensitivity.

83. The sensor device of claim 79, wherein the bolus estimator includes a memory to store information.

84. The sensor device of claim 83, wherein the memory stores one or more databases used in estimating the bolus amount, said one or more databases being selected from the group consisting of a user history, a food library, a drug library, and a bar code library.

85. The sensor device of claim 81, wherein the bolus estimator provides intelligent therapy to the user based on the one or more databases.

86. The sensor device of claim 79, further including a housing to contain the bolus estimator and the monitor.

87. The sensor device of claim 86, wherein the housing is selected from the group consisting of a keychain, a watch, a piece of jewelry, an accessory card, a Smartphone, and a key fob.

88. The sensor device of claim 86, further including a receptacle formed in the housing, wherein the receptacle receives a fluid from the user, and wherein the sensor electronics measures the analyte level of the user from the fluid.
